(19) United States
(12) Patent Application Publication
(10) Pub. No.: US 2026/0257043 A1
(43) Pub. Date: Sep. 3, 2026

(54) ADJUSTABLE VENTRICULAR DRAINAGE CATHETER CLAMPING SYSTEM FOR NEUROSURGERY

(71) Applicant: Guangzhou Institute of Cancer Research,the Affiliated Cancer Hospital,Guangzhou Medical University, Guangzhou (CN)

(72) Inventors: HAILIN ZHAO, Guangzhou (CN); LULU WANG, Guangzhou (CN); PING TANG, Guangzhou (CN)

(21) Appl. No.: 19/293,583

(22) Filed: Aug. 7, 2025

Related U.S. Application Data
(63) Continuation of application No. PCT/CN2025/080006, filed on Feb. 28, 2025.

(51) Int. Cl. A61M 25/02

(52) U.S. Cl. CPC A61M 25/02; A61M 2025/024; A61M 2025/0266; A61M 2025/028; A61M 2209/088

(57) ABSTRACT

An adjustable ventricular drainage catheter clamping system for neurosurgery includes a fixing box, an adjustment assembly, a fixing assembly, and a clamping assembly; the fixing box has a rectangular structure and is internally provided with a sliding groove and a recess located on a front side of the fixing box; the adjustment assembly includes a unidirectional threaded rod rotatably connected to the sliding groove; a sliding block is disposed on the unidirectional threaded rod, and a connecting post is connected to a bottom of the sliding block; and an adjustment box is fixedly connected to a bottom of the connecting post; and the fixing assembly includes a snap-fit slot located on a side of the recess, and a second spring; and an end of the second spring is fixedly connected to a clamping plate, and two sides of the clamping plate are fixedly connected with guide rods for positional limitation.

### CROSS-REFERENCE TO RELATED APPLICATIONS

The present application is a Continuation of PCT/CN2025/080006, filed on Feb. 28, 2025 which are hereby incorporated by reference in its entirety.

### TECHNICAL FIELD

The present disclosure relates to the field of drainage catheter clamping apparatus, and in particular to an adjustable ventricular drainage catheter clamping system for neurosurgery.

### BACKGROUND

Ventricular drainage is a procedure that involves inserting a drainage catheter having multiple side holes into the cerebral ventricle via a burr hole or a trephination puncture in the skull, with a distal end connected to a sterile drainage bottle to drain cerebrospinal fluid out of a body. It is a commonly used emergency treatment in neurosurgery for life-saving purposes. Ventricular drainage is indicated for relieving elevated intracranial pressure caused by cerebrospinal fluid circulation disorders, especially when brain herniation is impending or has just occurred. Ventricular drainage can help prevent or mitigate the development of brain herniation, thus playing a significant role in saving lives. During the ventricular drainage procedure, a drainage catheter is often used, and needs to be secured by clamping devices during operation. The clamping device is known as ventricular drainage catheter clamping apparatus. With the rapid advancement of science and technology, the clamping apparatus has undergone various technical improvements. However, in current practice, although positioning and catheter arrangement can be achieved by the clamping apparatus, it is inconvenient to adjust a clamping position or orientation when a direction of the drainage catheter changes or when a clamping location is suboptimal, which may easily cause the drainage catheter to be bent or compressed at the clamping apparatus, hindering smooth operation. Moreover, the existing clamping apparatus is usually complicated to adjust and secure, further affecting operation and use effect in clinical use.

### SUMMARY

A technical problem to be solved by the present disclosure is to overcome the above technical defects.

In order to solve the above problems, the present disclosure provides an adjustable ventricular drainage catheter clamping system for neurosurgery, including a fixing box, an adjustment assembly, a fixing assembly, and a clamping assembly;

the fixing box has a rectangular structure and is internally provided with a sliding groove and a recess, the recess is located on a front side of the fixing box; and the adjustment assembly is positioned in the sliding groove;

the adjustment assembly includes a unidirectional threaded rod, the unidirectional threaded rod is rotatably connected to the sliding groove, and an end of the unidirectional threaded rod extends through the sliding groove; a sliding block is disposed on the unidirectional threaded rod, and a connecting post is connected to a bottom of the sliding block; and an adjustment box is fixedly connected to a bottom of the connecting post, and the adjustment box is provided with two clamping cylinders; and

the fixing assembly is disposed in the recess; the fixing assembly includes a snap-fit slot, the snap-fit slot is located on a side of the recess; the fixing assembly further includes a second spring, the second spring is fixedly connected to the recess; and an end of the second spring is fixedly connected to a clamping plate, and two sides of the clamping plate are fixedly connected with guide rods for positional limitation.

Further, the end of the unidirectional threaded rod is connected to a rotating wheel for adjusting rotation.

Further, the adjustment box is internally provided with a placement chamber, a threaded rod is disposed inside the placement chamber, and a second sliding block is mounted on the threaded rod; and a connecting groove is fixedly connected with a front side of the second sliding block, a spring is disposed in the connecting groove, a limiting block is fixedly connected with an end of the spring, and connecting rods are fixedly connected with both sides of the limiting block.

Further, the two connecting rods are respectively fixedly connected to the clamping cylinders, and are configured to clamp and secure the clamping cylinders.

Further, a second snap-fit slot that mates with the snap-fit slot is formed on a front side of the clamping plate for positional limitation and clamping.

Further, the clamping assembly includes the clamping cylinders; each clamping cylinder includes a fixed base, a safety locking mechanism, and an anti-drop barb; and a medical-grade pressure-sensitive adhesive patch is used as an adhesive layer on the fixed base and is configured to secure the clamping cylinders to a patient’s skin or a bed sheet. The safety locking mechanism uses a latch or a twist lock to prevent unintentional release of clamping force. The anti-drop barb is configured to automatically secure the inserted drainage catheter and require removal at a specific angle to disengage.

Compared with the prior art, the present disclosure has the advantages:

In the present disclosure, a fixing box is arranged, an adjustment assembly, a fixing assembly, and a clamping assembly are arranged on the fixing box, and an unidirectional threaded rod of the adjustment assembly is rotatably connected to the sliding groove, such that the movement of the sliding block on the unidirectional threaded rod drives the adjustment box to move, and the clamping cylinders are driven to move.

The fixing assembly includes a snap-fit slot, and the snap-fit slot is located on a side of the recess. A clamping plate is adjusted using the elastic force of a second spring, and guide rods on both sides of the clamping plate are used for positional limitation.

### BRIEF DESCRIPTION OF DRAWINGS

FIG. 1 is a first perspective view of an adjustable ventricular drainage catheter clamping system for neurosurgery according to the present disclosure.

FIG. 2 is a second perspective view of an adjustable ventricular drainage catheter clamping system for neurosurgery according to the present disclosure.

FIG. 3 is a third perspective view of an adjustable ventricular drainage catheter clamping system for neurosurgery according to the present disclosure.

### REFERENCE NUMERALS IN THE ACCOMPANYING DRAWINGS

1. fixing box; 2. sliding groove; 3. recess; 4. unidirectional threaded rod; 5. sliding block; 6. connecting post; 7. adjustment box; 8. connecting rod; 9. clamping cylinder; 10. snap-fit slot; 11. second spring; 12. clamping plate; 13. guide rod; and 14. second snap-fit slot.

### DESCRIPTION OF EMBODIMENTS

The specific embodiments of the present disclosure will be further described below in conjunction with the accompanying drawings. Identical components are denoted by the same reference numerals in the drawings.

It should be noted that the terms “front,” “rear,” “left,” “right,” “top,” and “bottom” used in the following description refer to directions as illustrated in the drawings, and the terms “inner” and “outer” refer to directions toward or away from the geometric center of a specific component.

In order to facilitate clearer understanding of the content of the present disclosure, the technical solutions of embodiments of the present disclosure will be described below clearly and comprehensively in conjunction with accompanying drawings of the embodiments of the present disclosure.

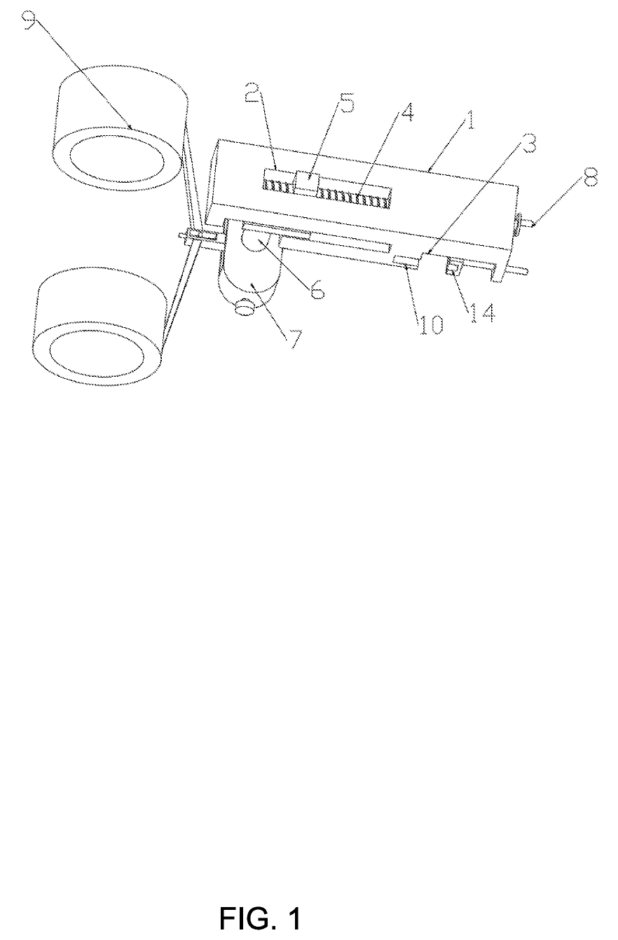

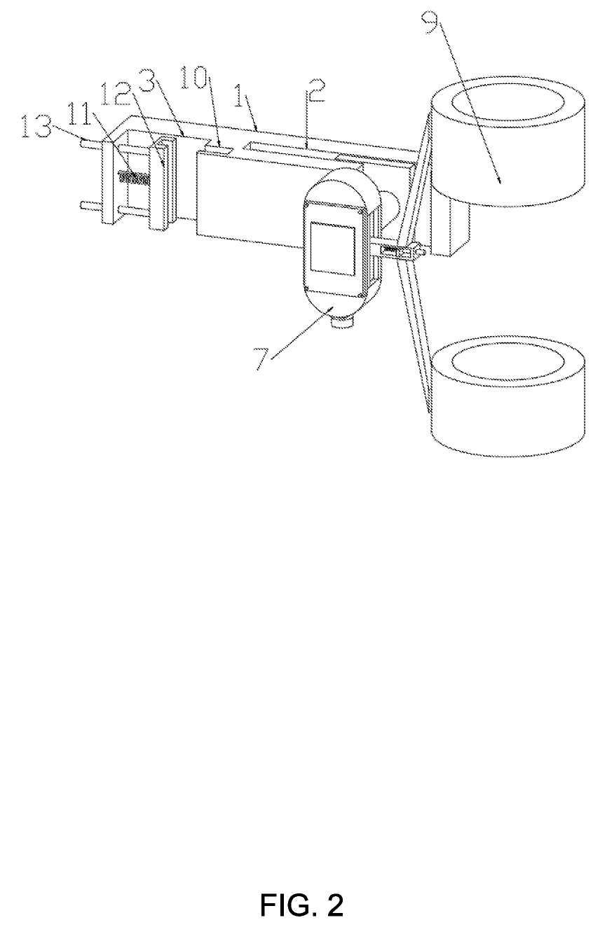

As shown in FIGS. 1-3, a neurosurgical adjustable ventricular drainage catheter holding system includes a fixing box 1, an adjustment assembly, a fixing assembly, and a clamping assembly; the fixing box 1 has a rectangular structure and is internally provided with a sliding groove 2 and a recess 3, the recess 3 is located on a front side of the fixing box 1; and the adjustment assembly is positioned in the sliding groove 2;

the adjustment assembly includes a unidirectional threaded rod 4, and the end of the unidirectional threaded rod 4 is connected to a rotating wheel 8 for adjusting rotation; the unidirectional threaded rod 4 is rotatably connected to the sliding groove 2, and an end of the unidirectional threaded rod extends through the sliding groove 2; a sliding block 5 is disposed on the unidirectional threaded rod 4, and a connecting post 6 is connected to a bottom of the sliding block 5; and an adjustment box 7 is fixedly connected to a bottom of the connecting post 6, and the adjustment box 7 is provided with two clamping cylinders 9; and

the fixing assembly is disposed in the recess 3; the fixing assembly includes a snap-fit slot 10, the snap-fit slot 10 is located on a side of the recess 3; the fixing assembly further includes a second spring 11, the second spring 11 is fixedly connected to the recess 3; and an end of the second spring is fixedly connected to a clamping plate 12, and two sides of the clamping plate 12 are fixedly connected with guide rods 13 for positional limitation.

The adjustment box 7 is internally provided with a placement chamber, a threaded rod is disposed inside the placement chamber, and a second sliding block is mounted on the threaded rod; and a connecting groove is fixedly connected with a front side of the second sliding block. A spring is disposed in the connecting groove, a limiting block is fixedly connected with an end of the spring, and connecting rods 8 are fixedly connected with both sides of the limiting block.

The two connecting rods 8 are respectively fixedly connected to the clamping cylinders 9, and are configured to clamp and secure the clamping cylinders 9. A second snap-fit slot 14 that mates with the snap-fit slot 10 is formed on a front side of the clamping plate 12 for positional limitation and clamping.

The clamping assembly includes the clamping cylinders 9. Each clamping cylinder 9 includes a fixed base, a safety locking mechanism, and an anti-drop barb; and a medical-grade pressure-sensitive adhesive patch is used as an adhesive layer on the fixed base and is configured to secure the clamping cylinders to a patient’s skin or a bed sheet. The safety locking mechanism uses a latch or a twist lock to prevent unintentional release of clamping force. The anti-drop barb is configured to automatically secure the inserted drainage catheter and require removal at a specific angle to disengage.

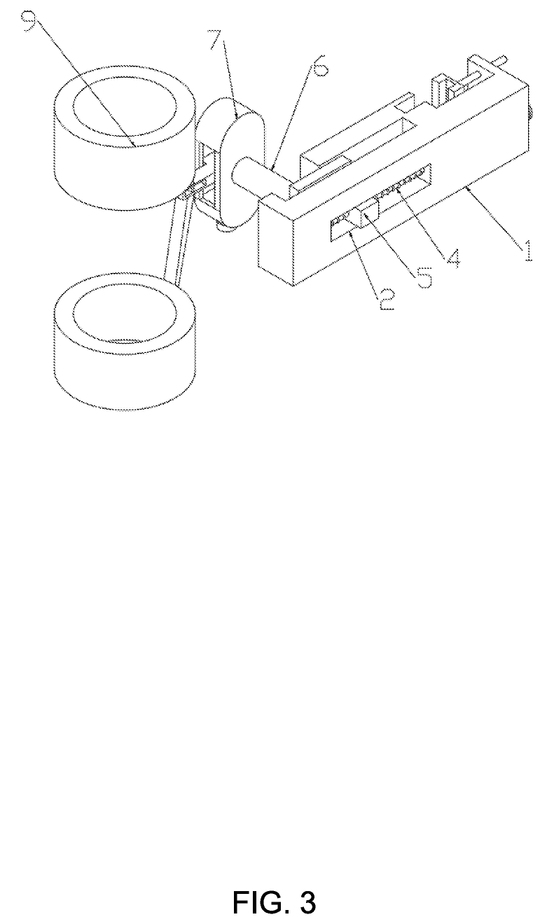

As shown in FIGS. 1 and 3, when in use, the unidirectional threaded rod 4 of the adjustment assembly is rotated to complete the operation. Specifically, when the rotating wheel 8 is rotated, the sliding block 5 in rotational connection with the unidirectional threaded rod 4 in turn drives the adjustment box 7 to move. In addition, the fixed base, the safety locking mechanism, and the anti-drop barb internally arranged in each of the two clamping cylinders 9 is configured to securely fix the drainage catheter. The fixed base, the safety locking mechanism, and the anti-drop barb all adopt the structures in the prior art.

The present disclosure and its embodiments are described in detail above, which is not limiting. The accompanying drawings illustrate only one embodiment of the present disclosure, and the actual structural configurations are not limited thereto. In summary, any structural modifications or alternative embodiments that may be devised by those skilled in the art, based on the teachings of the present disclosure and without departing from the spirit and scope of the present disclosure, shall fall within the scope of protection of the present disclosure.

What is claimed is:

1. An adjustable ventricular drainage catheter clamping system for neurosurgery, comprising a fixing box (1), an adjustment assembly, a fixing assembly, and a clamping assembly; wherein
the fixing box (1) has a rectangular structure and is internally provided with a sliding groove (2) and a recess (3), the recess (3) is located on a front side of the fixing box (1), and the adjustment assembly is positioned in the sliding groove (2);
the adjustment assembly comprises a unidirectional threaded rod (4); the unidirectional threaded rod (4) is rotatably connected to the sliding groove (2), and an end of the unidirectional threaded rod extends through the sliding groove (2); a sliding block (5) is disposed on the unidirectional threaded rod (4), and a connecting post (6) is connected to a bottom of the sliding block (5); and an adjustment box (7) is fixedly connected to a bottom of the connecting post (6), and the adjustment box (7) is provided with two clamping cylinders (9); and
the fixing assembly is disposed in the recess (3); the fixing assembly comprises a snap-fit slot (10), the snap-fit slot (10) is located on a side of the recess (3); the fixing assembly further comprises a second spring (11), the second spring (11) is fixedly connected to the recess (3); and an end of the second spring is fixedly connected to a clamping plate (12), and two sides of the clamping plate (12) are fixedly connected with guide rods (13) for positional limitation.

2. The adjustable ventricular drainage catheter clamping system for neurosurgery of claim 1, wherein the end of the unidirectional threaded rod (4) is connected to a rotating wheel (8) for adjusting rotation.

3. The adjustable ventricular drainage catheter clamping system for neurosurgery of claim 1, wherein the adjustment box (7) is internally provided with a placement chamber, a threaded rod is disposed inside the placement chamber, and a second sliding block is mounted on the threaded rod; and a connecting groove is fixedly connected with a front side of the second sliding block; and a spring is disposed in the connecting groove, a limiting block is fixedly connected with an end of the spring, and connecting rods (8) are fixedly connected with both sides of the limiting block.

4. The adjustable ventricular drainage catheter clamping system for neurosurgery of claim 3, wherein the two connecting rods (8) are respectively fixedly connected to the clamping cylinders (9) and are configured to clamp and secure the clamping cylinders.(9).

5. The adjustable ventricular drainage catheter clamping system for neurosurgery of claim 1, wherein a second snap-fit slot (14) that mates with the snap-fit slot (10) is formed on a front side of the clamping plate (12) for positional limitation and clamping.

6. The adjustable ventricular drainage catheter clamping system for neurosurgery of claim 4, wherein the clamping assembly comprises the clamping cylinders (9); each clamping cylinder (9) comprises a fixed base, a safety locking mechanism, and an anti-drop barb; a medical-grade pressure-sensitive adhesive patch is used as an adhesive layer on the fixed base and is configured to secure the clamping cylinders to a patient’s skin or a bed sheet; the safety locking mechanism uses a latch or a twist lock to prevent unintentional release of clamping force; and the anti-drop barb is configured to automatically secure the inserted drainage catheter and require removal at a defined angle to disengage.
